SEQUENCE LISTING (6 sequences)

SEQ ID NO: 1 - DNA, 689 bp, Sphingomonas
  16 S rDNA (1)..(689)<s300> DSM 162542004-02-24(1)..(689)
gaacgctggsggcatgcctaacacatgcaagtcgaacgaagtcttcggacttagtggcgcacgggtgcgtaacgcgtgggaatctgcccttgggtacggaataactcagagaaatttgtgctaataccgtataatgtcttcggaccaaagatttatcgcccaaggatgagcccgcgtaagattagctagttggtggggtaaaggcccaccaaggcgacgatctttagctggtctgagaggatgatcagccacactgggactgagacacggcccagactcctacgggaggcagcagtggggaatattggacaatgggcgaaagcctgatccagcaatgccgcgtgagtgatgaaggccctagggttgtaaagctcttttacccgggatgataatgacagtaccgggagaataagctccggctaactccgtgccagcagccgcggtaatacggagggagctagcgttgttcggaattactgggcgtaaagcgcgcgtaggcggtttttcaagtcagaggtgaaagcccggggctcaaccccggaattgcctttgaaactggaagacttgaatcttggagaggtcagtggaattccgagtgtagaggcgaaattcgtagatattcggaagaacaccagtggcgaaggcgactgactggacaagattgacgctgaggtgcgaaagcgtgggga

SEQ ID NO: 2 - DNA, 426 bp, Sphingomonas
  16 S rDNA (1)..(426)<s300> DSM 162572004-02-24(1)..(426)
gatcctggctcagaacgaacgctggcggcatgcctaacacatgcaagtcgaacgaagtcttcggacttagtggcgcacgggtgcgtaacgcgtgggaatctgcccttgggtacggaataactcagagaaatttgtgctaataccgtataatgacttcggtccaaagatttatcgcccaaggatgagcccgcgtaagattagctagttggtggggtaaaagcctaccaaggcgacgatctttagctggtctgagaggatgatcagccacactgggactgagacacggcccagactcctacgggaggcagcagtggggaatattggacaatgggcgaaagcctgatccagcaatgccgcgtgagtgatgaaggccctagggttgtaaagctcttttacccgggatgataatgacagtaccgggagaat

SEQ ID NO: 3 - DNA, 720 bp, Rhizobiales
  16 S rDNA (1)..(720)<s300> DSM2004-02-24(1)..(720)
acgctggcggcaggcttaacacatgcaagtcgaacggtctcttcggaggcagtggcagacgggtgagtaatgcatgggaatctaccgttctctacggaataactcagggaaacttgtgctaataccgtatacgcccttttggggaaagatttatcggagaatgatgagcccatgttggattagctagttggtagggtaaaggcctaccaaggcgacgatccatagctggtctgagaggatgatcagccacactgggactgagacacggcccagactcctacgggaggcagcagtggggaatattggacaatgggcgcaagcctgatccagccatgccgcgtgagtgatgaaggccctagggttgtaaagctctttcaccggtgaagataatgacggtaaccggagaagaagccccggctaacttcgtgccagcagccgcggtaatacgaagggggctagcgttgttcggatttactgggcgtaaagcgcacgtaggcggacttttaagtcaggggtgaaatcccggggctcaaccccggaactgcctttgatactggaagtcttgagtatggaagaggtaagtggaattgcgagtgtagaggtgaaattcgtagatattcgcaggaacaccagtggcgaaggcggcttactggtccattactgacgctgaggtgcgaaagcgtgggggagcaaacaggattagataccctggtagtccac

SEQ ID NO: 4 - DNA, 706 bp, Microbacterium
  16 S rDNA (1)..(706)<s300> DSM 162562004-02-24(1)..(706)
gaacgctggcggcgtgcttaacacatgcaagtcgaacgatgaagctggagcttgctctggtggaagagtggcgaacgggtgagtaacacgtgagtaacctgccccagactctgggataagcgctggaaacggcgtctaatactggatatgacccctacaggcatctgttgggggtggaaagatttatcggtctgggatgggctcgcggcctatcagctagatggtgaggtaacggctcaccatggcgacgacgggtagccggcctgagagggtgaccggccacactgggactgagacacggcccagactcctacgggaggcagcagtggggaatattgcacaatgggcgaaagcctgatgcagcaacgccgcgtgagggatgactgccttcgggttgtaaacctcttttagtagggaagaagcgaaagtgacggtacctgcagaaaaagcaccggctaactacgtgccagcagccgcggtaatacgtagggtgcaagcgttgtccggaattattgggcgtaaagagctcgtaggcggcttgtcgcgtctgctgtgaaaacccgaggctcaacctcgggcctgcagtgggtacgggcaggctagagtgcggtaggggagattggaattcctggtgtagcggtggaatgcgcagatatcaggaggaacaccgatggcgaaggcagatctctgggccgctactgacgct

SEQ ID NO: 5 - DNA, 392 bp, Rhizobium
  16 S rDNA (1)..(392)<s300> DSM 162532004-02-24(1)..(392)
tcctggctcagaacgaacgctggcggcaggcttaacacatgcaagtcgagcgccccgcaaggggagcggcagacgggtgagtaacgcgtgggaatctaccgagccctgcggaatagctccgggaaactggaattaataccgcatacgccctacgggggaaagatttatcggggtttgatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccctagggttgtaaagctctttcac

SEQ ID NO: 6 - DNA, 476 bp, Alcaligenes
  16 S rDNA (1)..(476)<s300> DSM 162522004-02-24(1)..(476)
tcctggctcagattgaacgctagcgggatgccttacacatgcaagtcgaacggcagcacggacttcggtctggtggcgagtggcgaacgggtgagtaatgtatcggaacgtgcctagtagcgggggataactacgcgaaagcgtagctaataccgcatacgccctacgggggaaagcaggggatcgcaagaccttgcactattagagcggccgatatcggattagctagttggtggggtaacggctcaccaaggcgacgatccgtagctggtttgagaggacgaccagccacactgggactgagacacggcccagactcctacgggaggcagcagtggggaattttggacaatgggggaaaccctgatccagccatcccgcgtgtgcgatgaaggccttcgggttgtaaagcacttttggcaggaaagaaacgtcatgggctaataccccgtgaaactgacggtacctgcagaata

(19) United States
(12) Patent Application Publication
(10) Pub. No.: US 2007/0292579 A1
(43) Pub. Date: Dec. 20, 2007

(54) Micro-organism for decontaminating fumonisins and its use, method for decontaminating fumonisins, and feed additive containing said micro-oragnism

(76) Inventors: Gerd Schatzmayr, Vienna (AT); Martin Taubel, Ternitz (AT); Elisavet Vekiru, Vienna (AT); Eva-Maria Binder, Tulln (AT)

Correspondence Address: JACOBSON HOLMAN PLLC, 400 SEVENTH STREET N.W., SUITE 600, WASHINGTON, DC, 20004, US

(21) Appl. No.: 11/798,700

(22) Filed: May 16, 2007

Related U.S. Application Data
(63) Continuation of application No. PCT/AT05/00453, filed on Nov. 15, 2005.

(30) Foreign Application Priority Data
Nov. 16, 2004 (AT) A 1912/2004

(51) Int. Cl. A23L 1/015; C12N 1/16; C12N 1/20

(52) U.S. Cl. 426/532; 435/252.1; 435/255.1

(57) ABSTRACT

The invention relates to a micro-organism for decontaminating fumonisins and fumonisin derivatives and to the use of bacteria or yeasts, alone or in a combination of two or more strains for decontaminating fumonisins and fumonisin derivatives in foodstuffs and/or feed. The invention also relates to a method for decontaminating fumonisins and fumonisin derivatives with the aid of a micro-organism and to a feed additive for inactivating mycotoxins, in particular fumonisins and fumonisin derivatives. The invention relates to a micro-organism for decontaminating fumonisins and fumonisin derivatives and to the use of bacteria or yeasts, alone or in a combination of two or more strains for decontaminating fumonisins and fumonisin derivatives in foodstuffs and/or feed. The invention also relates to a method for decontaminating fumonisins and fumonisin derivatives with the aid of a micro-organism and to a feed additive for inactivating mycotoxins, in particular fumonisins and fumonisin derivatives.

This is a continuation of PCT/AT05/000453 filed Nov. 15, 2005 and published in German

### FIELD OF THE INVENTION

The present invention relates to a microorganism for decontaminating fumonisins and fumonisin derivatives and to the use of bacteria or yeasts, alone or in combination of two or more strains, for detoxifying fumonisins and fumonisin derivatives in foods and/or feeds, a method for decontaminating fumonisins and fumonisin derivatives using a microorganism, and a feed additive for inactivating mycotoxins, in particular fumonisins and fumonisin derivatives.

### PRIOR ART

Mycotoxins, which comprise a plurality of different toxins, constitute an increasing problem in the modern food and feed industries, since a plurality of plants which are subsequently processed to foods or feeds or directly fed to animals are infested with the most diverse toxins in the most diverse concentrations such that, in addition to the fact that the respective toxin has to be detected, an efficient and innocuous method for detoxifying or degrading the respective toxins will have to be applied or found.

An approach to obtain toxin-free plants has been the attempt to grow so-called transgenic plants, which are resistant to specific toxins, or to obtain food products by the aid of “genetically modified” plants, which food products are free of such toxins due to the resistance of the respective plants to the former.

In addition to being extremely complex and complicated, this approach has raised controversies in many countries throughout the world, and attempts have been made to find other ways of decontaminating plants.

Toxins frequently encountered especially in maize and leading to serious impairments after the consumption of the same are fumonisins and fumonisin derivatives, which can be degraded in laboratory tests by already known microorganisms, for which, however, no microorganisms have yet been discovered, which are able to perform such degradations in different toxin concentrations and in different nutrient environments. Known microorganisms, moreover, require quite considerable periods of time exceeding 24 hours for such degradations, so that the use of such microorganisms on an industrial or commercial scale would be unpractical.

Another problem in connection with mycotoxins in foods or feeds resides in that, due to the fact that an ever increasing amount of mixed feeds or mixed foods containing a plurality of cereals or cereal species are produced, several mycotoxins will, at the same time, occur in one and the same food or feed product such that a useful or selective degradation of the former appears necessary. Recent studies have, moreover, revealed that toxins may exhibit combinatory interactions among themselves, which would further intensify the noxious effects of the individual toxins. In 1996 Harvey, for instance, reported on synergistic effects of fumonisins and deoxynivalenol in pigs. In addition, it is assumed that the majority of toxins which may, for instance, be contained in feeds will lead to immunosuppressive effects in animals, which are ascribed to the parallel occurrence of mycotoxins.

### SUMMARY OF THE INVENTION

The present invention aims to provide microorganisms for decontaminating fumonisins and fumonisin derivatives, which are able to degrade the toxin extremely rapidly, on the one hand, and, in addition to such a rapid degradation, perform said degradation even in the presence of the most diverse nutrient concentrations, on the other hand. Finally, the present invention aims to provide a microorganism or combinations of microorganisms, which are able to degrade, in addition to fumonisins, also other toxins alone or in combination so as to obtain a toxin-free feed, particularly when using the most diverse feed plants.

To solve this object, a microorganism for decontaminating fumonisins and fumonisin derivatives is provided according to the present invention, wherein detoxifying bacteria or yeasts selected from the strains DSM 16254 and DSM 15257, assignable to the taxon Sphingomonadaceae, strain DSM 16255, assignable to the taxon Rhizobiales, strain DSM 16256, assignable to the taxon Microbacteriaceae, strain DSM 16253, assignable to the taxon Rhizobiaceae, strain DSM 16252, assignable to the taxon Alcaligenaceae, and Pichia sp. DSM 16562, are used, which convert fumonisins enzymatically into deaminated metabolites in a single-step or multi-step reaction. The above-mentioned microorganisms are able to not only convert fumonisins enzymatically into deaminated metabolites in a single-step or multi-step reaction, but do this within extremely short periods of time and even in the presence of complex environments such as, e.g., feeds or foods, i.e. in the presence of several or most diverse carbon sources and, in particular, in the presence of a nutrient oversupply.

In detail, the microorganisms can be briefly described as follows. Strain DSM 16254 is to be assigned to the taxon Sphingomonadaceae after partial sequencing of the 16S rDNA with the forward primer 27 (sequence length 689 bp). The partial 16S rDNA sequence has the following base sequence:

| | |
|---|---|
| 1 | AGGCGCTGGS GGCATGCCTA ACACATGCAA GTCGAACGAA GTCTTCGGAC TTAGTGGCGC |
| 61 | ACGGGTGCGT AACGCGTGGG AATCTGCCCT TGGGTACGGA ATAACTCAGA GAAATTTGTG |
| 121 | CTAATACCGT ATAATGTCTT CGGACCAAAG ATTTATCGCC CAAGGATGAG CCCGCGTAAG |
| 181 | ATTAGCTAGT TGGTGGGGTA AAGGCCCACC AAGGCGACGA TCTTTAGCTG GTCTGAGAGG |
| 241 | ATGATCAGCC ACACTGGGAC TGAGACACGG CCCAGACTCC TACGGGAGGC AGCAGTGGGG |
| 301 | AATATTGGAC AATGGGCGAA AGCCTGATCC AGCAATGCCG CGTGAGTGAT GAAGGCCCTA |
| 361 | GGGTTGTAAA GCTCTTTTAC CCGGGATGAT AATGACAGTA CCGGGAGAAT AAGCTCCGGC |
| 421 | TAACTCCGTG CCAGCAGCCG CGGTAATACG GAGGGAGCTA GCGTTGTTCG GAATTACTGG |
| 481 | GCGTAAAGCG CGCGTAGGCG GTTTTTCAAG TCAGAGGTGA AAGCCCGGGG CTCAACCCCG |
| 541 | GAATTGCCTT TGAAACTGGA AGACTTGAAT CTTGGAGAGG TCAGTGGAAT TCCGAGTGTA |
| 601 | GAGGCGAAAT TCGTAGATAT TCGGAAGAAC ACCAGTGGCG AAGGCGACTG ACTGGACAAG |
| 661 | ATTGACGCTG AGGTGCGAAA GCGTGGGGA |

wherein the microorganism is gram-negative and forms small rods occurring, above all, in single cells and partially forming filamentous chain structures.

Strain DSM 16257 likewise belongs to the taxon Sphingomonadaceae after partial sequencing of 16S rDNA (with the reverse primer 30, obtained sequence length 426 bp). The following sequence results:

| | |
|---|---|
| 1 | GATCCTGGCT CAGAACGAAC GCTGGCGGCA TGCCTAACAC ATGCAAGTCG AACGAAGTCT |
| 61 | TCGGACTTAG TGGCGCACGG GTGCGTAACG CGTGGGAATC TGCCCTTGGG TACGGAATAA |
| 121 | CTCAGAGAAA TTTGTGCTAA TACCGTATAA TGACTTCGGT CCAAAGATTT ATCGCCCAAG |
| 181 | GATGAGCCCG CGTAAGATTA GCTAGTTGGT GGGGTAAAAG CCTACCAAGG CGACGATCTT |
| 241 | TAGCTGGTCT GAGAGGATGA TCAGCCACAC TGGGACTGAG ACACGGCCCA GACTCCTACG |
| 301 | GGAGGCAGCA GTGGGGAATA TTGGACAATG GGCGAAAGCC TGATCCAGCA ATGCCGCGTG |
| 361 | AGTGATGAAG GCCCTAGGGT TGTAAAGCTC TTTTACCCGG GATGATAATG ACAGTACCGG |
| 421 | GAGAAT |

This microorganism forms small rods which, for the major part, are arranged in long, filamentous cell structures.

DSM 16255 after partial sequencing of the 16S rDNA with the forward primer 27 produces the following, 720-bp-long sequence:

| | |
|---|---|
| 1 | ACGCTGGCGG CAGGCTTAAC ACATGCAAGT CGAACGGTCT CTTCGGAGGC AGTGGCAGAC |
| 61 | GGGTGAGTAA TGCATGGGAA TCTACCGTTC TCTACGGAAT AACTCAGGGA AACTTGTGCT |
| 121 | AATACCGTAT ACGCCCTTTT GGGGAAAGAT TTATCGGAGA ATGATGAGCC CATGTTGGAT |
| 181 | TAGCTAGTTG GTAGGGTAAA GGCCTACCAA GGCGACGATC CATAGCTGGT CTGAGAGGAT |
| 241 | GATCAGCCAC ACTGGGACTG AGACACGGCC CAGACTCCTA CGGGAGGCAG CAGTGGGGAA |
| 301 | TATTGGACAA TGGGCGCAAG CCTGATCCAG CCATGCCGCG TGAGTGATGA AGGCCCTAGG |
| 361 | GTTGTAAAGC TCTTTCACCG GTGAAGATAA TGACGGTAAC CGGAGAAGAA GCCCCGGCTA |
| 421 | ACTTCGTGCC AGCAGCCGCG GTAATACGAA GGGGGCTAGC GTTGTTCGGA TTTACTGGGC |
| 481 | GTAAAGCGCA CGTAGGCGGA CTTTTAAGTC AGGGGTGAAA TCCCGGGGCT CAACCCCGGA |
| 541 | ACTGCCTTTG ATACTGGAAG TCTTGAGTAT GGAAGAGGTA AGTGGAATTG CGAGTGTAGA |
| 601 | GGTGAAATTC GTAGATATTC GCAGGAACAC CAGTGGCGAA GGCGGCTTAC TGGTCCATTA |
| 661 | CTGACGCTGA GGTGCGAAAG CGTGGGGGAG CAAACAGGAT TAGATACCCT GGTAGTCCAC |

The microorganism belongs to the taxon Rhizobiales and is gram-negative, forming small rods primarily in single cells.

The microorganism DSM 15256 is assignable to the taxon Microbacteriaceae. Partial sequencing of the 16S rDNA with the forward primer 27 yields the following, 706-pb-long sequence:

| | |
|---|---|
| 1 | GAACGCTGGC GGCGTGCTTA ACACATGCAA GTCGAACGAT GAAGCTGGAG CTTGCTCTGG |
| 61 | TGGAAGAGTG GCGAACGGGT GAGTAACACG TGAGTAACCT GCCCCAGACT CTGGGATAAG |
| 121 | CGCTGGAAAC GGCGTCTAAT ACTGGATATG ACCCCTACAG GCATCTGTTG GGGGTGGAAA |
| 181 | GATTTATCGG TCTGGGATGG GCTCGCGGCC TATCAGCTAG ATGGTGAGGT AACGGCTCAC |
| 241 | CATGGCGACG ACGGGTAGCC GGCCTGAGAG GGTGACCGGC CACACTGGGA CTGAGACACG |
| 301 | GCCCAGACTC CTACGGGAGG CAGCAGTGGG GAATATTGCA CAATGGGCGA AAGCCTGATG |
| 361 | CAGCAACGCC GCGTGAGGGA TGACTGCCTT CGGGTTGTAA ACCTCTTTTA GTAGGGAAGA |
| 421 | AGCGAAAGTG ACGGTACCTG CAGAAAAAGC ACCGGCTAAC TACGTGCCAG CAGCCGCGGT |
| 481 | AATACGTAGG GTGCAAGCGT TGTCCGGAAT TATTGGGCGT AAAGAGCTCG TAGGCGGCTT |
| 541 | GTCGCGTCTG CTGTGAAAAC CCGAGGCTCA ACCTCGGGCC TGCAGTGGGT ACGGGCAGGC |
| 601 | TAGAGTGCGG TAGGGGAGAT TGGAATTCCT GGTGTAGCGG TGGAATGCGC AGATATCAGG |
| 661 | AGGAACACCG ATGGCGAAGG CAGATCTCTG GGCCGCTACT GACGCT |

The microorganism is gram-positive and comprises small, short rods partially arranged in chain-like cell aggregates.

DSM 16253, after partial sequencing of the 16S rDNA (reverse primer 530, sequence length 392 bp), belongs to the taxon Rhizobiaceae. The sequence reads as follows:

| | |
|---|---|
| 1 | TCCTGGCTCA GAACGAACGC TGGCGGCAGG CTTAACACAT GCAAGTCGAG CGCCCCGCAA |
| 61 | GGGGAGCGGC AGACGGGTGA GTAACGCGTG GGAATCTACC GAGCCCTGCG GAATAGCTCC |
| 121 | GGGAAACTGG AATTAATACC GCATACGCCC TACGGGGGAA AGATTTATCG GGGTTTGATG |
| 181 | AGCCCGCGTT GGATTAGCTA GTTGGTGGGG TAAAGGCCTA CCAAGGCGAC GATCCATAGC |
| 241 | TGGTCTGAGA GGATGATCAG CCACATTGGG ACTGAGACAC GGCCCAAACT CCTACGGGAG |
| 301 | GCAGCAGTGG GGAATATTGG ACAATGGGCG CAAGCCTGAT CCAGCCATGC CGCGTGAGTG |
| 361 | ATGAAGGCCC TAGGGTTGTA AAGCTCTTTC AC |

The microorganism is gram-negative and comprises small rods occurring, above all, as single cells.

The microorganism DSM 15252 is assignable to the taxon Alcaligenaceae. Partial sequencing of the 16S rDNA produces a 476-bp-long DNA fragment having the following nucleotide sequence:

| | |
|---|---|
| 1 | TCCTGGCTCA GATTGAACGC TAGCGGGATG CCTTACACAT GCAAGTCGAA CGGCAGCACG |
| 61 | GACTTCGGTC TGGTGGCGAG TGGCGAACGG GTGAGTAATG TATCGGAACG TGCCTAGTAG |
| 121 | CGGGGGATAA CTACGCGAAA GCGTAGCTAA TACCGCATAC GCCCTACGGG GGAAAGCAGG |
| 181 | GGATCGCAAG ACCTTGCACT ATTAGAGCGG CCGATATCGG ATTAGCTAGT TGGTGGGGTA |
| 241 | ACGGCTCACC AAGGCGACGA TCCGTAGCTG GTTTGAGAGG ACGACCAGCC ACACTGGGAC |
| 301 | TGAGACACGG CCCAGACTCC TACGGGAGGC AGCAGTGGGG AATTTTGGAC AATGGGGGAA |
| 361 | ACCCTGATCC AGCCATCCCG CGTGTGCGAT GAAGGCCTTC GGGTTGTAAA GCACTTTTGG |
| 421 | CAGGAAAGAA ACGTCATGGG CTAATACCCC GTGAAACTGA CGGTACCTGC AGAATA |

The microorganism is gram-negative and comprises small, straight rods partially occurring in lumpy, multiple-cell aggregates.

DSM 16562, i.e. Pichia sp., shows relatively small, oval yeast cells occurring individually rather than in cell aggregates.

In detail, it could be demonstrated that all of the microorganisms, although relatively distinct from one another, in addition to the ability of rapidly detoxifying have in common the property of rapidly and reliably performing such a detoxification of fumonisins even in complex environments.

According to a further development of the invention, the bacteria or yeasts are stabilized in the form of powders, liquids or gels so as to provide a stable product capable of being applied at any time for the respective purpose.

As in correspondence with a further development of the invention, the bacteria or yeasts are used as cell-free extracts or crude extracts such that the usable product of microorganisms will be rapidly and reliably producible.

In order to remove toxins from foods or feeds as completely as possible, it is feasible according to a further development of the invention to detoxify by the aid of the microorganisms according to the present invention, in addition to fumonisin and fumonisin derivates, at least one further mycotoxin selected from zearalenones, aflatoxins or ochratoxins. In detail, it has turned out that the microorganisms according to the invention are able to detoxify at least one further toxin, such a detoxification being as rapidly and efficiently achievable as the detoxification of fumonisins. By providing said microorganisms, it is, thus, feasible without any further additive to completely degrade a plurality of toxins contained in a food or feed product, particularly in a food or feed mixture, and hence make available a high-quality, toxin-free food or feed product.

The invention also relates to the use of bacteria or yeasts, alone or in combination of two or more strains, selected from the strains DSM 16254 and DSM 15257, assignable to the taxon Sphingomonadaceae, strain DSM 16255, assignable to the taxon Rhizobiales, strain DSM 16256, assignable to the taxon Microbacteriaceae, strain DSM 16253, assignable to the taxon Rhizobiaceae, strain DSM 16252, assignable to the taxon Alcaligenaceae, and Pichia sp. DSM 16562, for detoxifying fumonisins and fumonisin derivatives in foods and/or feeds. By using the microorganisms according to the present invention, it is not only feasible to achieve a complete detoxification of fumonisins and fumonisin derivatives in foods or feeds, but, in addition to the fact that said microorganisms are capable of detoxification in media providing an excess carbon supply, said microorganisms are able to perform such detoxifications within extremely short periods of time. By using the above-mentioned microorganisms, it is, moreover, feasible to detoxify, in addition to fumonisins, at least one further mycotoxin selected from zearalenones, aflatoxins or ochratoxins. Such a use will safeguard, particularly in mixed feeds or mixed cereal products for human consumption, that several toxins present in grain will be safely and rapidly degraded by the aid of the microorganisms according to the invention.

In order to further complete said degradation, the invention contemplates the use of mixed cultures from bacteria and/or yeasts for detoxifying mycotoxins. The use of mixed cultures enables the selective attack against a plurality of present toxins that are simultaneously present in one and the same food or feed product or feed mixture and, hence, the achievement of a complete decontamination of the same. Furthermore, such a use has for the first time enabled the safe avoidance or prevention of the occurrence of undesired synergistic effects caused by the simultaneous occurrence of several mycotoxin species.

The use of the microorganisms according to the invention, moreover, enables the degradation of extremely low concentrations of the most diverse mycotoxins and, in particular, 100 μg/kg to 500 mg/kg, preferably 250 μg/kg to 25 mg/kg, fumonisins and fumonisin derivatives, 10 μg/kg to 10 mg/kg, preferably 40 μg/kg to 2 mg/kg, zearalenones and zearalenone derivatives, 1 μg/kg to 2 mg/kg, preferably 10 μg/kg to 750 μg/kg, aflatoxins, 1 μg/kg to 2 mg/kg, preferably 5 μg/kg to 500 μg/kg, ochratoxins, whereby it is ensured, in addition to the fact that the decontamination of the most diverse toxins has become feasible by the use of the microorganisms according to the invention, that also extremely low concentrations of said toxins will be attacked and degraded, what has so far been difficult if not impossible with conventional microorganisms.

In order to achieve as complete a detoxification as possible of all toxins contained, for instance, in a mixed feed, the use according to the invention is further developed to the extent that a combination or mixed culture additionally containing at least one further bacterium or yeast selected from Sphingomonas sp. DSM 14170 and DSM 14167, Stenotrophomonas nitritreducens DSM 14168, Stenotrophomonas sp. DSM 14169, Ralstonia eutropha DSM 14171, Eubacterium sp. DSM 14197, Trichosporon mycotoxinivorans DSM 14153, Cryptococcus sp. DSM 14154, Rhodotorula yarrowii DSM 14155, Trichosporon mucoides DSM 14156, Trichosporon dulcitum DSM 14162 or Eubacterium DSM 11798 is used for detoxifying mycotoxins, in particular fumonisins and fumonisin derivatives, zearalenones or zearalenone derivatives, ochratoxins, trichothecenes and/or aflatoxins. By using a combination or mixed culture additionally containing at least one further bacterium or yeast suitable for the degradation of, in particular, trichothecenes, zearalenones or zearalenone derivatives, aflatoxins or ochratoxins, it has become possible, in addition to the detoxifying effect of the microorganisms according to the present invention, i.e. the degradation of fumonisins, to expand their degradative ability in respect to other toxins to the extent that the selected use of several microorganisms enables the rapid and complete degradation of all toxins possibly present in a feed product, collectively and independently of one another.

In a method for decontaminating fumonisins and fumonisin derivatives using a microorganism according to the present invention, it is essentially proceeded in a manner that fumonisins in fodder with specific germ counts are enzymatically degraded into deaminated metabolites in a single-step or multi-step reaction. According to a further development, said detoxification is preferably carried out under aqueous conditions in minimal medium or complex environments with excess nutrient supply and carbon sources. Such a method control allows for the use of the microorganisms according to the present invention in methods in which the detoxification is performed directly within the feed, without taking into account the amount of carbon available to the microorganisms. This is of particular relevance in that the major portion of the hitherto known microorganisms are merely able to show their detoxifying effects in minimal medium or in environments having no elevated carbon supply, for which reason most of the known microorganisms are unsuitable for direct use in foods and feeds because of an extensive carbon supply.

According to a preferred further development, the method is controlled in a manner as to be completed within 15 min to 12 h and, in particular, 15 min to 2 h. By such a method control, it will, on the one hand, be ensured that all of the mycotoxins contained in the food or feed product, in particular fumonisins, will have been degraded and, on the other hand, it will be feasible to not only degrade mycotoxins, but carry out said degradation within such a short time as to enable the application of such a method on a large scale rather than just on a laboratory scale.

If, as in correspondence with a further development of the method according to the present invention, a combination or mixed culture additionally containing at least one further bacterium or yeast selected from Sphingomonas sp. DSM 14170 and DSM 14167, Stenotrophomonas nitritreducens DSM 14168, Stenotrophomonas sp. DSM 14169, Ralstonia eutropha DSM 14171, Eubacterium sp. DSM 14197, Trichosporon mycotoxinivorans DSM 14153, Cryptococcus sp. DSM 14154, Rhodotorula yarrowii DSM 14155, Trichosporon mucoides DSM 14156, Trichosporon dulcitum DSM 14162 or Eubacterium DSM 11798 is used for detoxifying mycotoxins, in particular fumonisins and fumonisin derivatives, zearalenones and zearalenone derivatives, ochratoxins, trichothecenes and/or aflatoxins is used, the method, in addition to the degradation of fumonisins and fumonisin derivatives and the degradation of the mycotoxins that are able to be additionally degraded by the microorganisms according to the invention, will be controlled in a manner as to achieve the complete decontamination of foods and/or feeds by the use of a selective choice of microorganisms.

In order to reliably complete said decontamination, a further development of the invention contemplates that, for decontaminating foods and/or feeds, the microorganisms are mixed with said foods and/or feeds each in amounts ranging from 0.01% by weight to 1.5% by weight and, in particular, 0.05% by weight to 0.7% by weight.

The invention finally comprises a feed additive for inactivating mycotoxins, in particular fumonisins and fumonisin derivatives, which is characterized in that said feed additive contains a microorganism according to any one of claims 1 to 4 at a germ count of from 2×10⁸/kg feed additive to 2×10¹⁵/kg feed additive and, in particular, 1×10⁹/kg feed additive to 5×10¹²/kg feed additive. By using feed additives containing said microorganisms at germ counts of from 2×10⁸/kg feed additive to 2×10¹⁵/kg feed additive, it is ensured that a complete decontamination of all of the fumonisins and fumonisin derivatives capable of being degraded by the microorganisms according to the invention will actually be effected and that, in addition, also any further toxins capable of being degraded by the microorganisms according to the invention will actually be degraded.

In order to expand said degradation to mycotoxins that can be degraded only partially or incompletely by the microorganisms according to the invention, the feed additive is further developed to the extent as to additionally contain at least one further bacterium or yeast selected from Sphingomonas sp. DSM 14170 and DSM 14167, Stenotrophomonas nitritreducens DSM 14168, Stenotrophomonas sp. DSM 14169, Ralstonia eutropha DSM 14171, Eubacterium sp. DSM 14197, Trichosporon mycotoxinivorans DSM 14153, Cryptococcus sp. DSM 14154, Rhodotorula yarrowii DSM 14155, Trichosporon mucoides DSM 14156, Trichosporon dulcitum DSM 14162 or Eubacterium DSM 11798 for detoxifying mycotoxins, in particular fumonisins and fumonisin derivatives, zearalenones and zearalenone derivatives, ochratoxins, trichothecenes and/or aflatoxins. By a selective combination of several microorganisms, the complete degradation of all toxins contained in one and the same feed product will, thus, be feasible so as to safely avoid, in particular, the synergistic effect of several toxins in a food or feed product.

As in correspondence with a further development of the invention, feed additives according to the present invention are suitable for the inactivation of fumonisins B1, B2, B3 and fumonisin derivatives, zearalenone, zearalenol, zearalenone glycosides, aflatoxins B1, B2, G1, G2, M1, M1, deoxynivalenol (DON), T-2 toxin, HT-2 toxin, nivalenol, monoacetoxyscirpenol, diacetoxyscirpenol, trichodermol, verrucarin, rorodin, acetyl deoxynivalenol, isotrichodermin, hydroxyisotrichodermin, calonectrin, T-2 tetraol, T-2 triol, deacetylneosolaniol, neosolaniol, acetylneosolaniol, sporotrichiol, trichotriol, sambucinol and culmorin and/or ochratoxins A, B, C, D in a feed product or in the digestive tract of an animal.

### PREFERRED EMBODIMENTS OF THE INVENTION

In the following, the invention will be explained in more detail by way of examples, Example 1 showing the time course of the degradation of fumonisin B1 at a constant toxin concentration in minimal medium, Example 2 showing the degradation of fumonisin B1 at different toxin concentrations, Example 3 showing the degradation of fumonisin B1 in complex media, Example 4 showing the degradation of fumonisin B1 in foods and feeds, Example 5 showing the degradation of ochratoxin using the microorganisms according to the invention, and Example 6 illustrating feeding tests using a microorganism mixture according to the present invention.

### Example 1

### Degradation or Detoxification of Fumonisin B in Minimal Medium at a Toxin Concentration of 2 mg/L Fumonisin B1

The tests were carried out using the microorganisms DSM 16254 and DSM 16257 as well as, for reasons of comparison, the strain Exophiala spinifera DSM 1217.

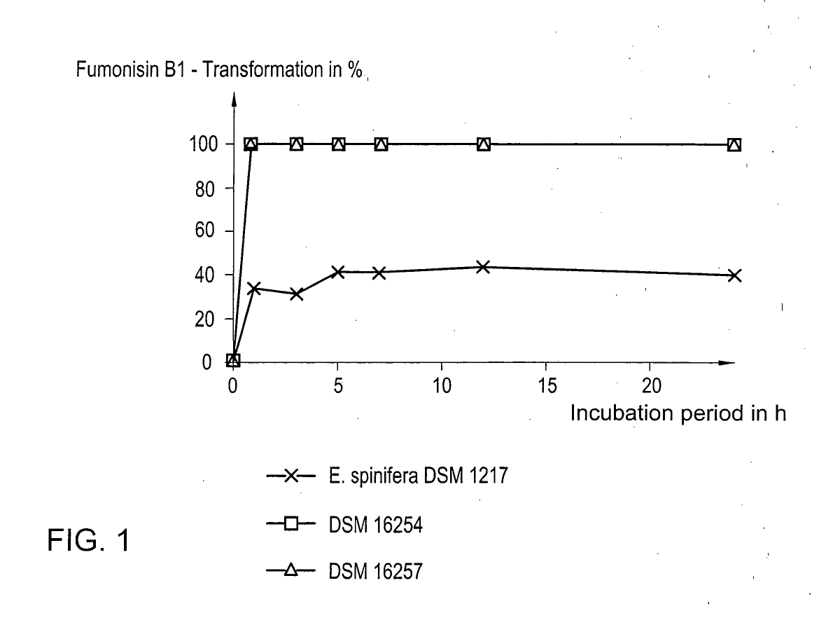

In all cases, the incubation took place at 25° C. under aerobic conditions. The cultivation of the microorganisms was carried out in the presence of 50 mg/l fumonisin B1 in common cultivation medium in order to enable an eventually possible induction of the fumonisin-B1-detoxifying enzymes. From FIG. 1 it is apparent that the strains DSM 16254 and DSM 16257 have transformed fumonisin B1 by 100% already after 1 h of incubation, while the comparative yeast strain E. spinifera was able to transform no more than 41% of the toxin after an incubation period of 24 h. The microorganisms according to the present invention, thus, not only are able to extremely rapidly detoxify fumonisin B1 in minimal medium, but such a detoxification will also occur 100%.

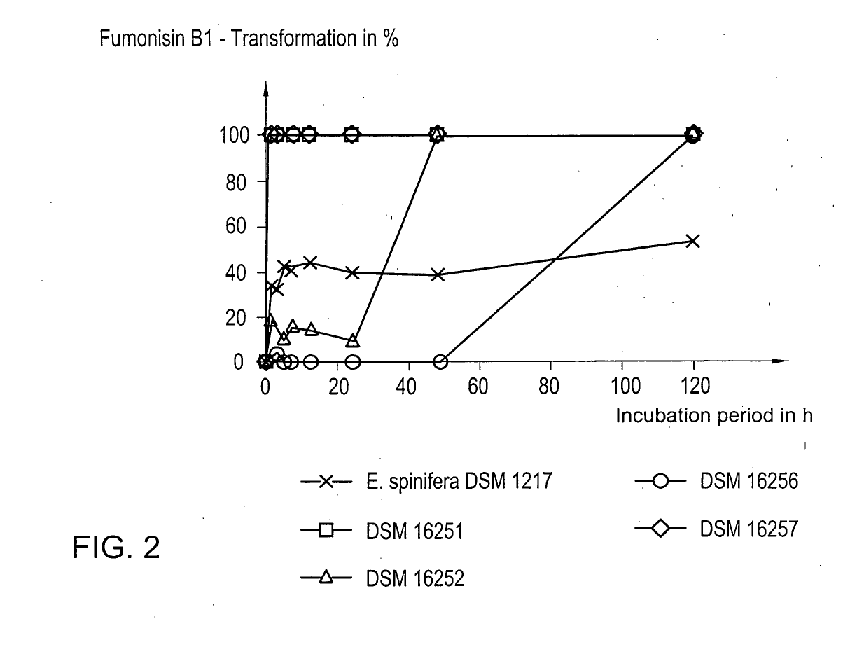

FIG. 2 shows the transformation over time in the same test assay, i.e. minimal medium and toxin concentration of 2 mg/l, for the strains DSM 16254, DSM 16256, DSM 16252, DSM 16257 as well as the yeast strain E. spinifera DSM 1217 by comparison. These degradation tests have clearly revealed that the microorganisms according to the invention are degraded extremely rapidly and in many cases, namely DSM 16254, DSM 16257, DSM 16252, DSM 16256, even 100%, which was impossible with the comparative microorganism DSM 1217.

### Example 2

### Degradation of Fumonisin B1 at Different Toxin Concentrations

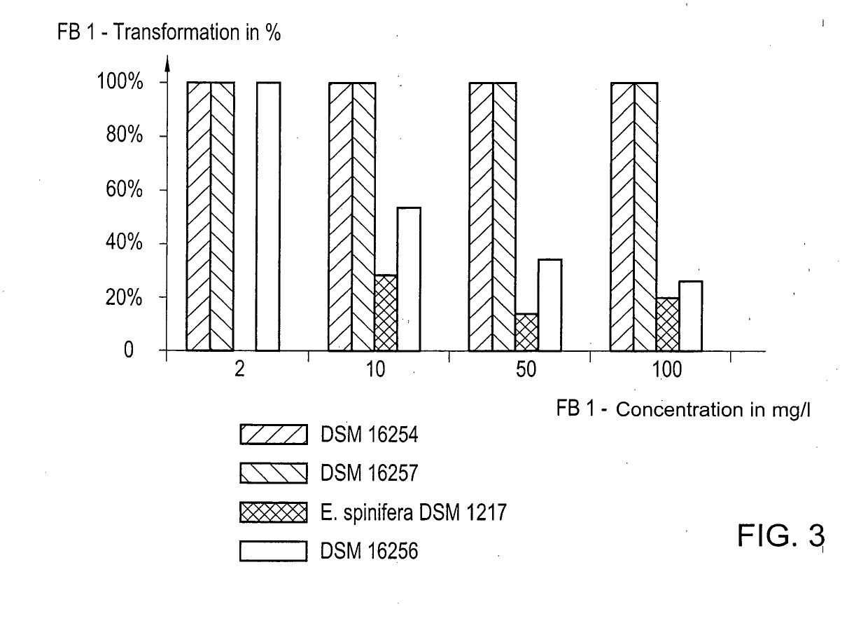

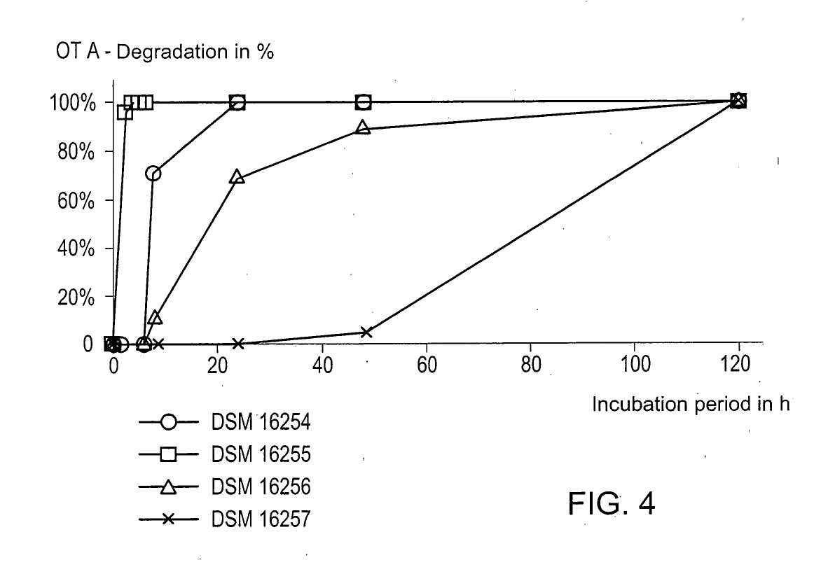

The tests were carried out with DSM 16254, DSM 16257 and DSM 16256 as well as with the yeast strain Exophiala spinifera DSM 1217 by comparison. The applied toxin concentrations were 2, 10, 50, 100 and 500 mg/l fumonisin B1. Incubation was effected under aerobic conditions at 25° C. The results are indicated after 5 h of incubation of the assays, since such incubation times constitute practise-relevant periods in respect to the detoxification of fumonisins in feeds. FIG. 3 shows the results of this test. The microorganism DSM 16254 was able to degrade fumonisin B1 100% in all concentration ranges, the microorganism DSM 16257 was merely able to reach a 96% degradation in a concentration range of 100 mg/l fumonisin B1, DSM 16256 enabled a 100% degradation at a concentration of 2 mg/l, a degradation of more than 50% at a concentration of 10 mg/l, a degradation of 35% and 25% at concentrations of 50 mg/l and 100 mg/l, respectively. The comparison with E. spinifera DSM 1217 demonstrated an extremely poor degradability for this microorganism, particularly at extremely low toxin concentrations, the best activity of DSM 1217 having occurred with 10 mg/l at a fumonisin B1 degradation rate of about 30%. From this comparison results that the microorganisms according to the present invention are superior to DSM 1217 in any concentration range and that a 100% degradation is possible, particularly at low toxin concentrations, what has not been possible so far with microorganisms according to the prior art.

### Example 3

### Degradation of Fumonisin B1 in Complex Medium

This test investigated the ability of the microorganisms to detoxify fumonisin B1 also in complex media in the presence of high nutrient concentrations. The cultivation of the microorganisms took place in a complex nutritive medium comprising 5 g/l peptone from meat extract and 3 g/1 meat extract, which was supplemented with two different concentrations of fumonisin B1, namely 10 mg/l and 100 mg/l. The determination of the transformation rates was effected by a comparison of the toxin contents in the assays at the beginning and at the end of a 72-hour-incubation at 25° C. under aerobic conditions. In both cases a 100% detoxification or 100% degradation of fumonisin B1 was obtained in the presence of 10 mg/l fumonisin B1 in the medium. Even in the presence of 100 mg/l fumonisin B1, a 100% detoxification was reached in both cases. This test clearly proved that the microorganisms according to the invention are suitable for the degradation of fumonisins in complex media, i.e. such with elevated nutrient supply.

### Example 4

### Degradation of Fumonisin B1 in Foods and Feeds

The microorganisms DSM 16254 and DSM 16257 were again used in an attempt to degrade toxin concentrations of 10 mg/l fumonisin B1 in beer, polenta and semolina. After having cultivated the microorganisms, the latter were harvested, resuspended in toxin-containing buffer solutions and subsequently incubated at once with the respective food or feed product. The degradation rate of fumonisin B1 was 100% in all cases, thus clearly proving that the microorganisms according to the invention are able to degrade fumonisins in feeds or foods 100%.

### Example 5

### Degradation of Other Mycotoxins by the Microorganisms According to the Invention

In this case, ochratoxin A was used as an exemplary mycotoxin. Strains DSM 16254, DSM 16255, DSM 16256 and DSM 16257 were used. The degradation of ochratoxin was carried out in the presence of 400 μg/l ochratoxin A in an aerobic buffer at 120 h of incubation. Strain DSM 16255 showed a 95% detoxification already after 2 h, after 24 h both the strains DSM 16254 and the strain DSM 16255 had detoxified ochratoxin A 100%, after 48 h a 90% detoxification could also be determined with DSM 16256, and after 120 h even the strain DSM 15257 had detoxified ochratoxin A 100%.

### Example 6

### Feeding Tests Using Combinations or Mixed Cultures of Different Microorganisms for the Complete Detoxification of Foods and Feeds Supplemented with Mycotoxins

Piglet Test I

In this test, strains DSM 16254 and DSM 14153 were used as additives. Each additive had an overall germ count of 1×10¹² KBE/kg additive. The test period was 42 days. The animals were subdivided into four groups of 24 animals each. The control group (KG) received uncontaminated standard feed without any feed additive. The toxin group (TG) received fodder supplemented with 500 ppb ochratoxin A, 250 ppb zearalenone and 1,500 ppb fumonisin B1. Test group 1 (VG1) and test group 2 (VG2) each received the same toxin-supplemented fodder, yet test group 1 with 0.5 kg additive and test group 2 with 1 kg additive. At the end of the test, the following results were achieved.

| | Final weight | Daily weight gain | FCR |
|---|---|---|---|
| KG | 24.3 kg | 434 g | 1.493 |
| TG | 22.0 kg | 380 g | 1.573 |
| VG1 | 23.4 kg | 412 g | 1.516 |
| VG2 | 24.7 kg | 443 g | 1.467 |

Piglet Test II

In this test, DSM 16254, DSM 11798 and DSM 14153 were used as additives. Each additive had an overall germ count of 2.5×10¹² KBE/kg additive. The test period was 42 days. The animals were subdivided into four groups of 19 animals each. The toxin group (TG) received fodder supplemented with 1.1 ppm deoxynivalenol and 2 ppm fumonisin B1, yet containing no additive. Test group 1 (VG1), test group 2 (VG2) and test group 3 (VG3) each received the same toxin-supplemented fodder, yet test group 1 with 0.5 kg additive, test group 2 with 1 kg additive and test group 3 with 2 kg additive. At the end of the test, the following results were achieved.

| | Final weight | Daily weight gain | FCR |
|---|---|---|---|
| TG | 22.90 kg | 359 g | 1.82 |
| VG1 | 26.45 kg | 442 g | 1.67 |
| VG2 | 27.10 kg | 463 g | 1.60 |
| VG3 | 28.55 kg | 485 g | 1.71 |

Piglet Test III

In this test, strain DSM 16254 was used as an additive. The additive had an overall germ count of 1×10¹¹ KBE/kg additive. The test period was 42 days. The animals were subdivided into two groups of 30 animals each. The toxin group (TG) received fodder supplemented with 4.5 ppm fumonisin B1. The test group received the same toxin-supplemented fodder, yet with 0.5 kg additive. At the end of the test, the following results were achieved.

| | | | Daily | Feed |
|---|---|---|---|---|
| | Initial | Final | weight | conversion |
| | weight | weight | gain | rate |
| Toxin group | 6.76 kg | 28.33 kg | 514 g | 2.21 |
| Test group | 6.88 kg | 30.56 kg | 564 g | 2.03 |

Broiler Test I

In this test, strain DSM 16254 was used as an additive. The additive had an overall germ count of 2.5×10¹¹ KBE/kg additive. The animals were subdivided into two groups of 140,000 animals each. The toxin group (TG) received fodder supplemented with 300 ppm aflatoxin and 2 ppm fumonisin. The test group received the same toxin-supplemented fodder, yet with 1 kg additive/ton fodder. At the end of the test, the following results were achieved.

| | Test group | Toxin group |
|---|---|---|
| Test week | Mortality [%] | Mortality [%] |
| 1 | 1.02 | 2.99 |
| 2 | 0.82 | 1.83 |
| 3 | 0.34 | 1.79 |
| 4 | 0.57 | 2.89 |
| 5 | 0.89 | 2.27 |
| 6 | 0.91 | 1.24 |
| 7 | 0.73 | 1.07 |
| 8 | 0.41 | 1.63 |
| Final weight [g] | 1720 | 1298 |

Broiler Test II

In this test, strains DSM 16254 and DSM 11798 were used as additives. Each additive had an overall germ count of 4×10¹¹ KBE/kg additive. The animals were subdivided into three groups of 260 animals each. The control group received uncontaminated fodder. The toxin group (TG) received fodder supplemented with 3.5 ppm fumonisin and 1.8 ppm t-2 toxin. The test group received the same toxin-supplemented fodder, yet with 1 kg additive/ton fodder. At the end of the test, the following results were achieved.

| | Control group | Test group | Toxin group |
|---|---|---|---|
| Weight gain | 1965.6 | 1952.4 | 1866.1 |
| Overal feed | 3983.9 | 4113.2 | 3894.0 |
| intake | | | |
| Feed conversion | 2.03 | 2.11 | 2.08 |
| rate | | | |

1: A microorganism for decontaminating fumonisins and fumonisin derivatives, wherein detoxifying bacteria or yeasts selected from the strains DSM 16254 and DSM 15257, assignable to the taxon Sphingomonadaceae, strain DSM 16255, assignable to the taxon Rhizobiales, strain DSM 16256, assignable to the taxon Microbacteriaceae, strain DSM 16253, assignable to the taxon Rhizobiaceae, strain DSM 16252, assignable to the taxon Alcaligenaceae, and Pichia sp. DSM 16562, are used, which convert fumonisins enzymatically into deaminated metabolites in a single-step or multi-step reaction.

2: The microorganism according to claim 1, wherein said bacteria or yeasts are stabilized in the form of powders, liquids or gels.

3: The microorganism according to claim 1, wherein said bacteria or yeasts are used as cell-free extracts or crude extracts.

4: The microorganism according to claim 1, wherein said bacteria or yeasts detoxify, in addition to fumonisin and fumonisin derivates, at least one further mycotoxin selected from zearalenones, aflatoxins or ochratoxins.

5: A use of bacteria or yeasts, alone or in combination of two or more strains, selected from the strains DSM 16254 and DSM 15257, assignable to the taxon Sphingomonadaceae, strain DSM 16255, assignable to the taxon Rhizobiales, strain DSM 16256, assignable to the taxon Microbacteriaceae, strain DSM 16253, assignable to the taxon Rhizobiaceae, strain DSM 16252, assignable to the taxon Alcaligenaceae, and Pichia sp. DSM 16562, for detoxifying fumonisins and fumonisin derivatives in foods and/or feeds.

6: The use according to claim 5, wherein said bacteria or yeasts, in addition, are employed for detoxifying at least one further mycotoxin selected from zearalenones, aflatoxins or ochratoxins.

7: The use according to claim 5, wherein mixed cultures of bacteria and/or yeasts are used for detoxifying mycotoxins.

8: The use according to claim 5, for decontaminating low mycotoxin concentrations and, in particular, 100 μg/kg to 500 mg/kg, preferably 250 μg/kg to 25 mg/kg, fumonisins and fumonisin derivatives, 10 μg/kg to 10 mg/kg, preferably 40 μg/kg to 2 mg/kg, zearalenones and zearalenone derivatives, 1 μg/kg to 2 mg/kg, preferably 10 μg/kg to 750 μg/kg, aflatoxins, 1 μg/kg to 2 mg/kg, preferably 5 μg/kg to 500 μg/kg, ochratoxins.

9: The use according to claim 5, wherein a combination or mixed culture additionally containing at least one further bacterium or yeast selected from Sphingomonas sp. DSM 14170 and DSM 14167, Stenotrophomonas nitritreducens DSM 14168, Stenotrophomonas sp. DSM 14169, Ralstonia eutropha DSM 14171, Eubacterium sp. DSM 14197, Trichosporon mycotoxinivorans DSM 14153, Cryptococcus sp. DSM 14154, Rhodotorula yarrowii DSM 14155, Trichosporon mucoides DSM 14156, Trichosporon dulcitum DSM 14162 or Eubacterium DSM 11798 is used for detoxifying mycotoxins, in particular fumonisins and fumonisin derivatives, zearalenones and zearalenone derivatives, ochratoxins, trichothecenes and/or aflatoxins, for detoxifying jointly occurring mycotoxins, in particular fumonisins and fumonisin derivatives, zearalenones and zearalenone derivatives, aflatoxins or ochratoxins.

10: The method for decontaminating fumonisins and fumonisin derivatives using a microorganism according to claim 1, comprising enzymatically converting fumonisins in fodder with a germ count of from 10³/g fodder to 10⁸/g fodder, in particular 2×10⁴/g fodder to 5×10⁶/g fodder, into deaminated metabolites in a single-step or multi-step reaction.

11: The method according to claim 10, wherein said detoxification is carried out under aqueous conditions in minimal medium or complex environments with excess nutrient supply and carbon sources.

12: The method according to claim 10, wherein it is carried out within 15 min to 12 h and, in particular, 15 min to 2 h.

13: The method according to claim 1, wherein at least one further mycotoxin selected from zearalenones, aflatoxins or ochratoxins is converted into a non-toxic degradation product.

14: The method according to claim 1, wherein a combination or mixed culture additionally containing at least one further bacterium or yeast selected from Sphingomonas sp. DSM 14170 and DSM 14167, Stenotrophomonas nitritreducens DSM 14168, Stenotrophomonas sp. DSM 14169, Ralstonia eutropha DSM 14171, Eubacterium sp. DSM 14197, Trichosporon mycotoxinivorans DSM 14153, Cryptococcus sp. DSM 14154, Rhodotorula yarrowii DSM 14155, Trichosporon mucoides DSM 14156, Trichosporon dulcitum DSM 14162 or Eubacterium DSM 11798 is used for detoxifying mycotoxins, in particular fumonisins and fumonisin derivatives, zearalenones and zearalenone derivatives, ochratoxins, trichothecenes and/or aflatoxins.

15: The method according to claim 1, wherein, for the decontamination of foods and/or feeds, the microorganisms are mixed with said foods and/or feeds each in amounts ranging from 0.01% by weight to 1.5% by weight and, in particular, 0.05% by weight to 0.7% by weight.

16: The feed additive for inactivating mycotoxins, in particular fumonisins and fumonisin derivatives, wherein said feed additive contains a microorganism according to claim 1 at a germ count of from 2×10⁸/kg feed additive to 2×10¹⁵/kg feed additive, in particular 1×10⁹/kg feed additive to 5×10¹²/kg feed additive.

17: The feed additive according to claim 16, wherein it additionally contains at least one further bacterium or yeast selected from Sphingomonas sp. DSM 14170 and DSM 14167, Stenotrophomonas nitritreducens DSM 14168, Stenotrophomonas sp. DSM 14169, Ralstonia eutropha DSM 14171, Eubacterium sp. DSM 14197, Trichosporon mycotoxinivorans DSM 14153, Cryptococcus sp. DSM 14154, Rhodotorula yarrowii DSM 14155, Trichosporon mucoides DSM 14156, Trichosporon dulcitum DSM 14162 or Eubacterium DSM 11798 for detoxifying mycotoxins, in particular fumonisins and fumonisin derivatives, zearalenones and zearalenone derivatives, ochratoxins, trichothecenes and/or aflatoxins.

18: The use of a feed additive according to claim 16 for inactivating fumonisins B1, B2, B3 and fumonisin derivatives, zearalenone and zearalenone derivatives, zearalenol, zearalenone glycosides, aflatoxins B1, B2, G1, G2, M1, M1, deoxynivalenol (DON), T-2 toxin, HT-2 toxin, nivalenol, monoacetoxyscirpenol, diacetoxyscirpenol, trichodermol, verrucarin, rorodin, acetyl deoxynivalenol, isotrichodermin, hydroxyisotrichodermin, calonectrin, T-2 tetraol, T-2 triol, deacetylneosolaniol, neosolaniol, acetylneosolaniol, sporotrichiol, trichotriol, sambucinol and culmorin and/or ochratoxins A, B, C, D in a feed product or in the digestive tract of an animal.
